(19) United States
(12) Patent Application Publication
(10) Pub. No.: US 2015/0352240 A1
(43) Pub. Date: Dec. 10, 2015

(54) Active Scrub with Baking Soda for Fridge Freshening and Food Preservation

(71) Applicant: Subhash Chander, Diamond Bar, CA (US)

(72) Inventor: Subhash Chander, Diamond Bar, CA (US)

(21) Appl. No.: 14/301,329

(22) Filed: Jun. 10, 2014

(51) Int. Cl. A61L 9/014

(52) U.S. Cl. CPC A61L 9/014

(57) ABSTRACT

Sodium Bi-carbonate, more commonly known as Baking Soda, is a chemical often used in killing odor or foul smell emanating from food kept in refrigerators and cooling cabinets and also as a preservative to extend the shelf life of food items. Invention titled “Active Scrub with Baking Soda for Fridge Freshening and Food Preservation” exploits these applications. This invention comprises—a process to scrub the environmental air contained inside the cooling chamber of refrigerators, freezers and cooling cabinets etc; with Baking Soda, using air ballast of the cold air circulation system of respective equipment; and—an attachment to accomplish this process. Such scrubbing facilitates chemical reaction of baking soda with the acidic contents of environmental air and the resultant change in pH in turn helps in killing the odor and extending the shelf-life of contents/food items stored there-in.

### BACKGROUND OF THE INVENTION

1. Field of Invention

Household Appliances—Refrigeration

2. Description of Related Art

Baking Soda, also known as sodium bicarbonate or sodium hydrogen carbonate or bicarbonate of soda has over sixty documented uses predominantly related to household cleansing, cooking, foods' preservation and hygiene etc. Killing of odor in the refrigerators and cooling cabinets and extending the self life are among its much practiced applications.

In refrigerators and cooling cabinets, baking soda is placed in open containers and intended purpose (de-odorizing and food preservation) is served by reaction of the air inside the compartment with the baking soda. Such contact initiates a chemical reaction with change in pH of the environmental air and eventually the de-odorizing and food preservation effects. However such contact is not initiated by any movement of air and/or baking soda and hence the reaction is passive.

U.S. Pat. No. 7,431,901 of Stiros et al envisaged sprinkle/disperse the powdered particles of baking soda in the cooling chamber of refrigerator/freezer/cooling cabinet. Such dispersal increases the likelihood of air, contained there-in; to come in contact with the baking soda and thus enhances the efficacy of the intended chemical reaction.

However, as of date, exploitation of this knowledge in daily life is nearly absent.

Link—This Non-Provisional Patent application is linked to Provisional Patent Application # 61/833,851 submitted on Jun. 11, 2013.

### SUMMARY

Baking Soda, also known as sodium bicarbonate, sodium hydrogen carbonate or bicarbonate of soda, is widely recommended for killing odor in the refrigerators and cooling cabinets and extending the shelf life of food items. As of now, this is accomplished by placing baking soda in open containers and the intended purpose is served by reaction of the air inside the compartment with the baking soda. However, such application is passive and hence has limited efficacy.

U.S. Pat. No. 7,431,901 of Stiros et al envisaged sprinkle/disperse the powdered particles of baking soda in the cooling chamber of refrigerator/freezer/cooling cabinet. Such dispersal increases the likelihood of air, contained there-in, to come in contact with the baking soda and thus enhances the efficacy of the intended chemical reaction.

However, as of date, exploitation of this knowledge in daily life is nearly absent.

Active Scrub with Baking Soda for Fridge Freshening and Food Preservation is a process to scrub the environmental air contained inside the cooling chamber of refrigerating appliances like refrigerators, freezers and cooling cabinets etc; with Baking Soda, using air ballast of the cold air circulation system of respective equipment. Such scrubbing is accomplished with the help of an attachment. It (scrubbing) increases the likelihood of acidic contents of environmental air to react with baking soda and the resultant change in pH yields the benefits of deodorization and food preservation. Attachment envisaged to accomplish this innovative process is a simple assembly and can be adapted in new as well as existing refrigerators/freezers/cooling cabinets that have an air circulation system.

While in case of new equipment it can be incorporated as an in-built feature of the respective refrigerator/freezer/cooling cabinet or as a stick-on/fastened/slid-in optional fixture, stick-on features can be easily provided in the existing refrigeration equipment to hold the ‘attachment’ and eventually harness the benefits of Active Scrub process.

Description of the Invention

Notes:

1. In describing the invention, references are drawn to the drawings in FIGS. 1.0 to 4.2, annexed to the Specifications; to explain the concepts and means to accomplish the intended purposes. However these drawings are highly simplified to explain the basics and are not drawn to scale and are schematic illustrations for conceptual understanding only.

2. Illustrations and shapes just include one of the several variants/choices available to exploit the invention.

3. The term ‘Refrigerator’ includes refrigerator, cooling cabinet and freezer etc. used for storage and preservation of food.

### BRIEF DESCRIPTION OF DRAWINGS ENCLOSED AS ANNEXURE

FIG. 1.0—components of Air Circulation System and flow of Circulation Air

FIG. 2.0—Air Circulation System in a ‘Refrigerator’

FIG. 3.1—Perforated Cartridge for keeping Baking Soda

FIG. 3.2—Frame of the Attachment envisaged for holding the Cartridge in desired position

FIG. 3.3—the Attachment (with Cartridge) as assembled

FIG. 4.1—Schematic Arrangement of Active Scrub System in a Refrigerator, depicting the Attachment held in position/attached inside the Refrigerator

FIG. 4.2—Active Scrub Attachment as assembled and attached to the air vent/outlet of the Refrigerator. It is an enlarged view of ASA depicted in FIG. 4.1.

Innovation titled ‘Active Scrub with Baking Soda for Fridge Freshening and Food Preservation’ is a process to scrub the environmental air contained inside the cooling chamber of refrigeration appliance like refrigerators, freezers and cooling cabinets etc; with Baking Soda, using air ballast of the cold air circulation system of respective equipment; and—an attachment to accomplish this process. Such scrubbing facilitates chemical reaction of baking soda with the acidic contents of environmental air and the resultant change in pH in turn helps in killing the odor and extending the shelf life of contents/food items stored there-in.

Basic components involved in the invention and their operation are described as follows:

Air Circulation System

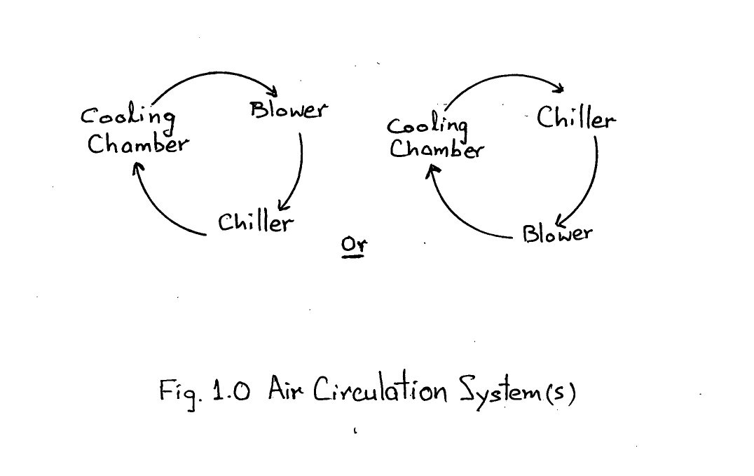

As is known, most of the refrigerators, freezers and cooling cabinets available in the market have an air circulation system which transfers the chill from refrigerant to the respective cooling chamber/s. This system works in close loop as depicted in the flow chart Annexed as FIG. 1.0.

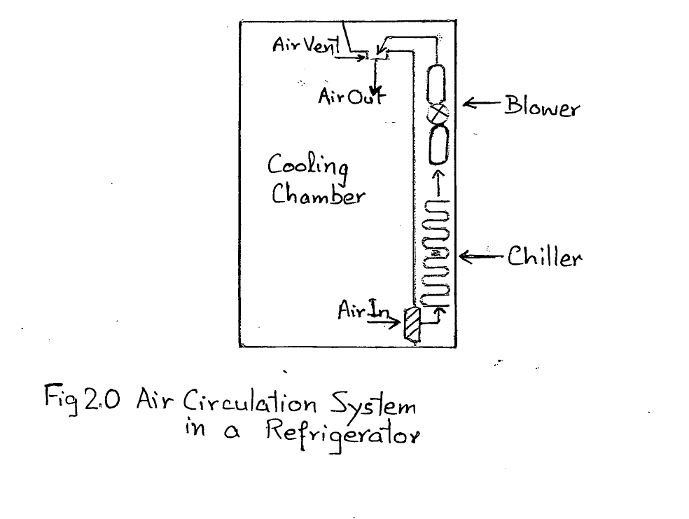

A blower is provided either before or after the chiller stack. This blower sucks environmental air of the cooling cabinet and feed it back as cold or chilled air, ready to cool the contents of the cabinet. Air inlet/s (suction/s) and outlet/s (vent/s) are provided in the cooling cabinet and are connected to each other through the blower and chiller located outside the cooling cabinet. (Refer FIG. 2.0 enclosed as Annexure).

The Attachment

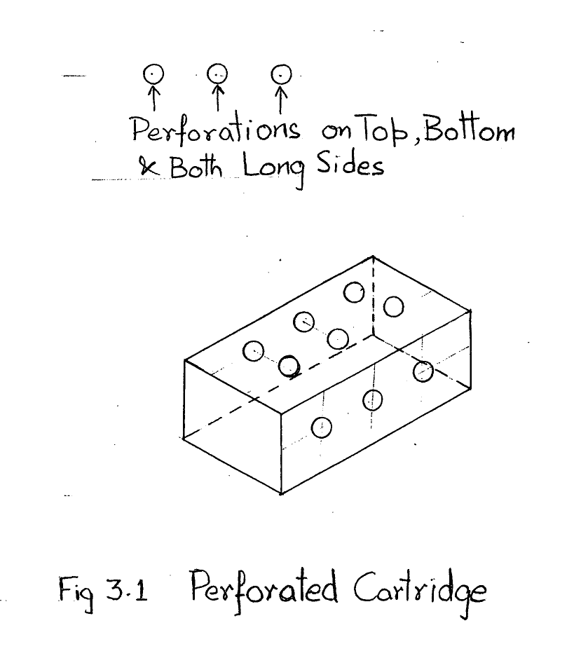

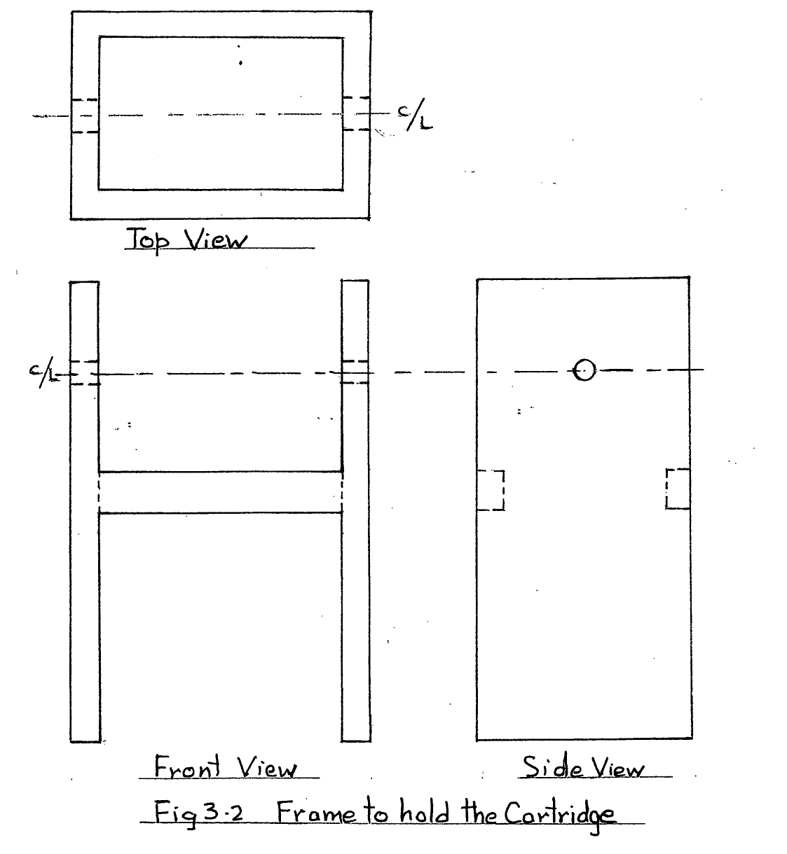

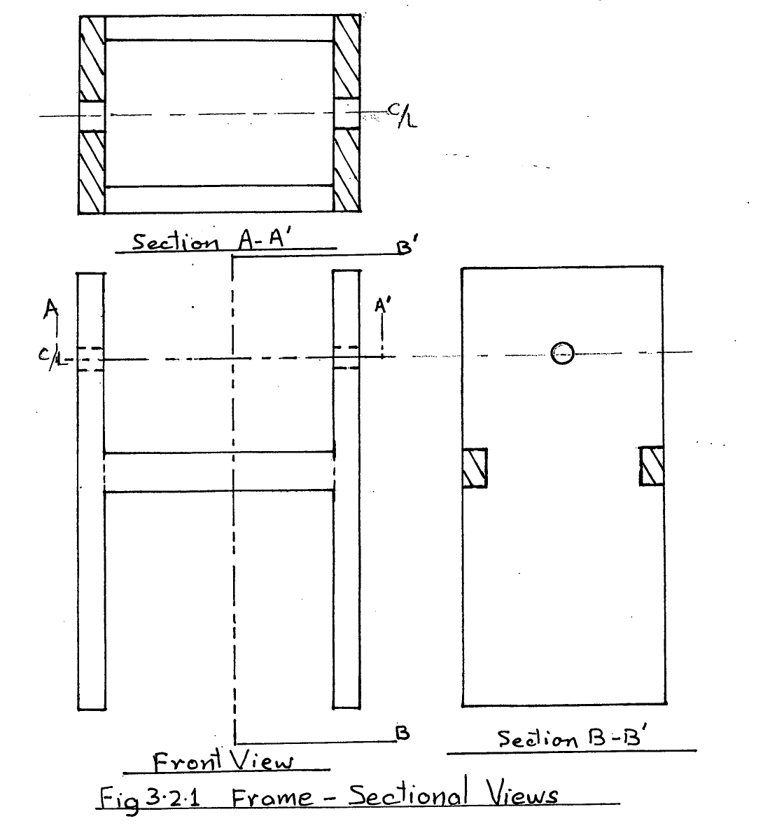

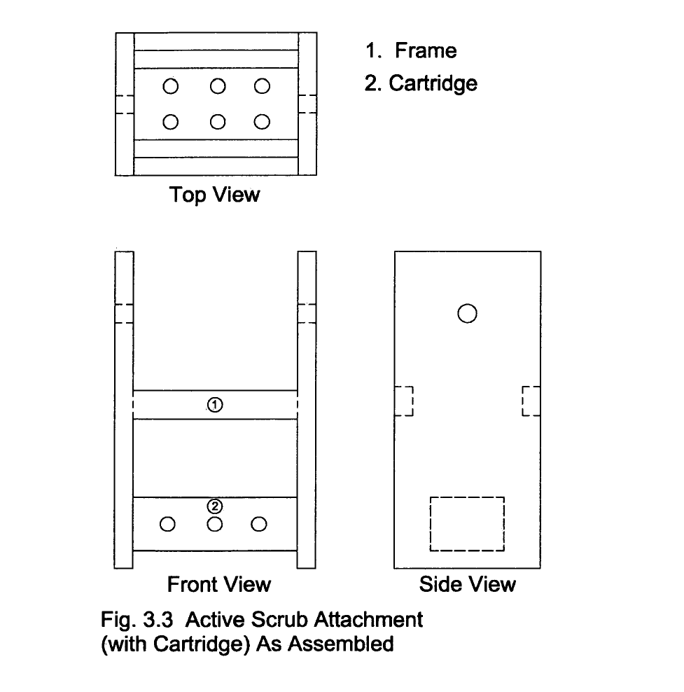

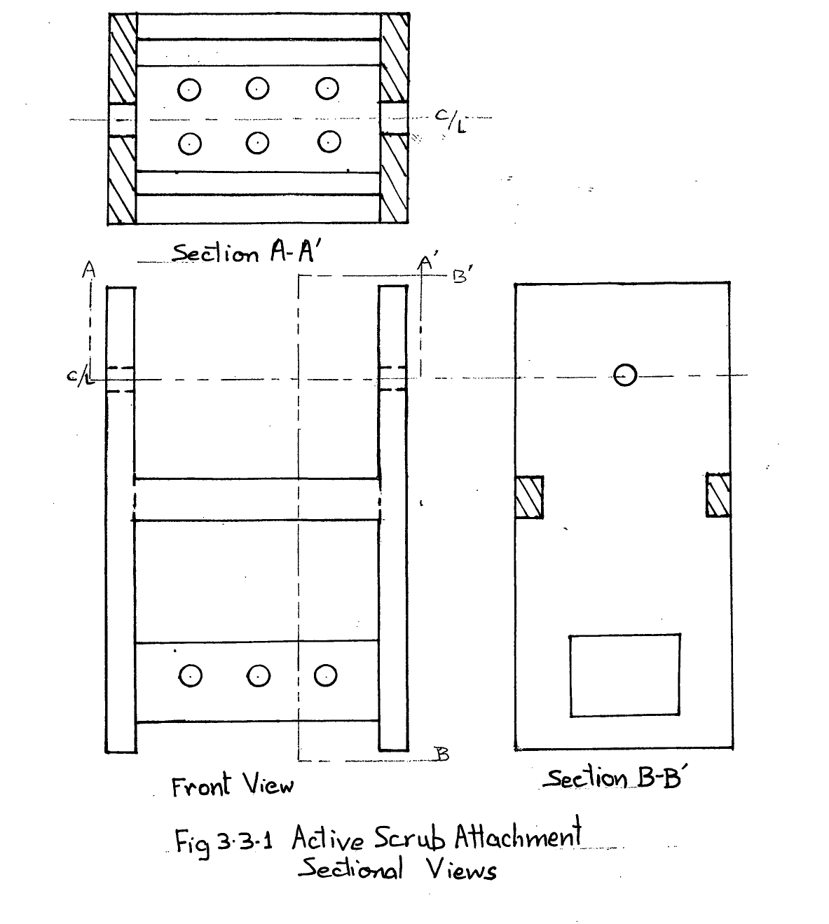

Attachment to accomplish the scrubbing of environmental air with baking soda comprises of:
A cartridge with perforated shell (Refer FIG. 3.1).
Complementing frame to hold/accommodate this cartridge (Refer FIG. 3.2).
Cartridge and Frame are assembled as depicted in FIG. 3.3 and thus forms the Attachment.
Baking soda is filled in the cartridge. The cartridge can be refillable or a onetime use/disposable component.

Baking soda is envisaged in its coarse form/shapes like, but not limited to; crystals, granules, chunks or nuggets, pellets, or tablets etc. Reason to use baking soda in coarse form is to avoid spills often associated when used as powder and ensure permeability for air to flow through the pack/cartridge.

Assembly and Positioning:

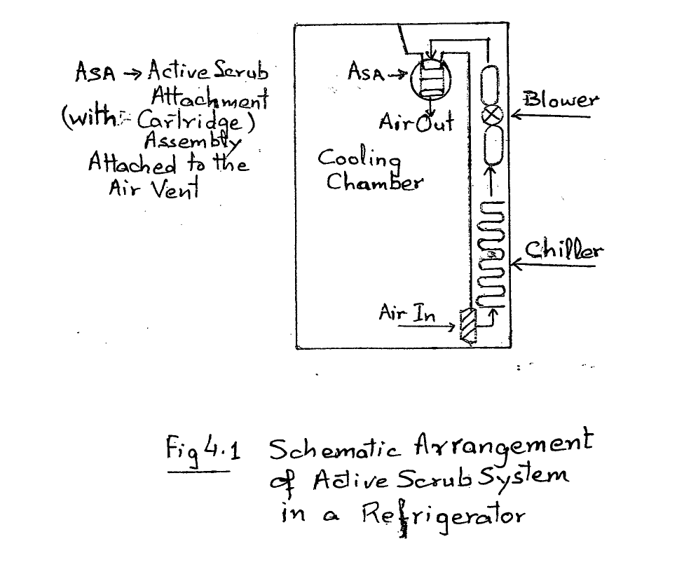

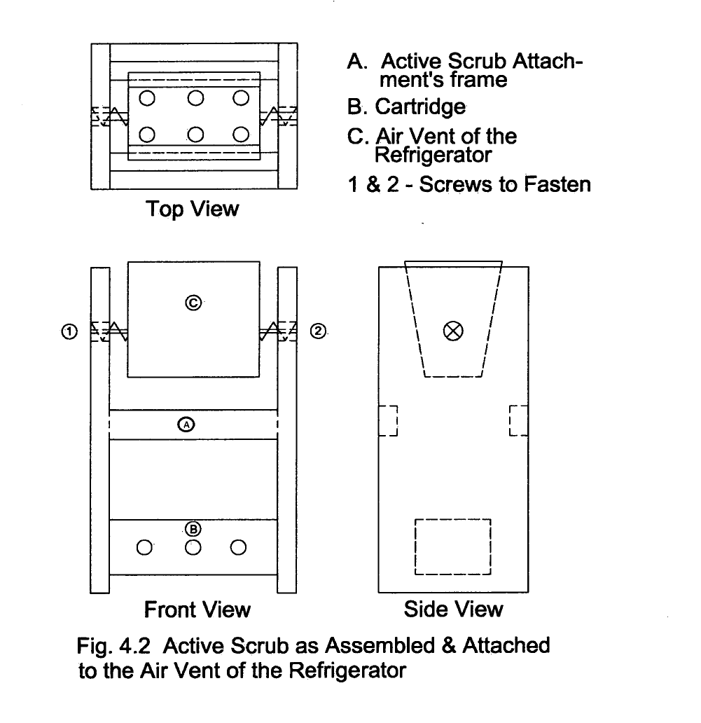

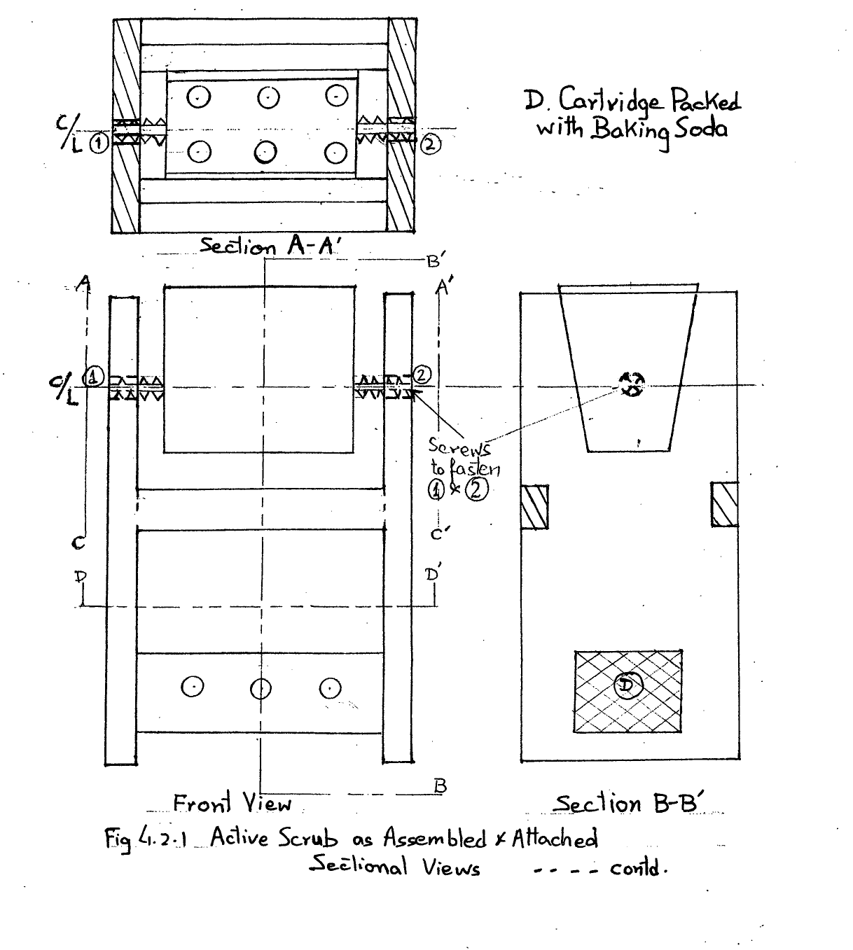

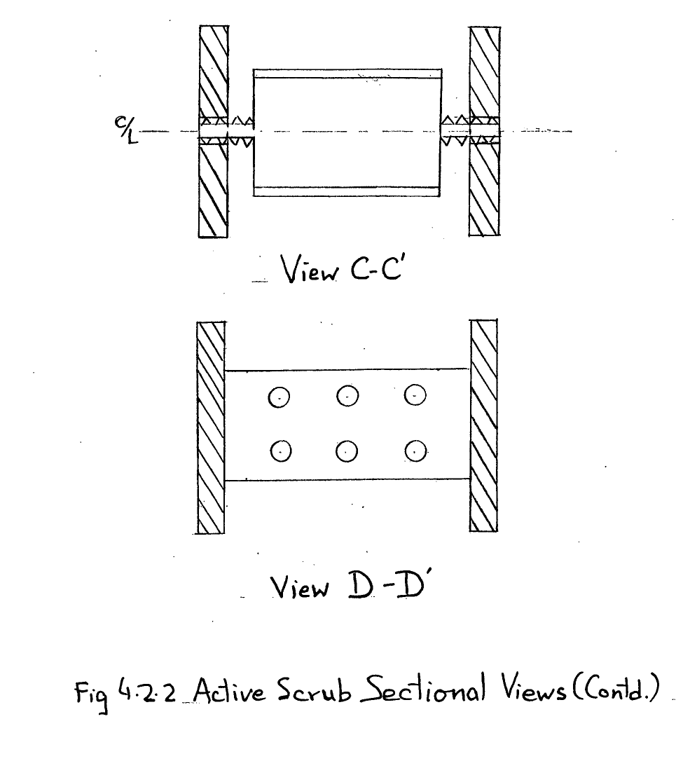

The Attachment (assembly of cartridge and frame) is attached or placed or hung or stuck close to the air vent or outlet of cold air circulation system of the refrigerator, as depicted in FIGS. 4.1 and 4.2 of the Annexure; with the help of suitable fasteners. The fitting/placement of attachment inside the refrigerator/freezer/cooling cabinet is done in such a way that the cartridge (which is slid into its place/grooves on the frame); lies across the direction of air flowing out of the air vent of cold air circulation system of the respective equipment. (Refer FIGS. 4.1 and 4.2).

Operation

When put to use, air of the air circulation system, emerging from the vent; impinges upon the baking soda. Such impingement results in scrubbing of air with the baking soda. Since environmental air of the cooling chamber is a part of the air circulation system, its scrubbing with baking soda neutralizes its acids. Such neutralization of acids change the pH of environmental air and the changed pH helps in killing the odor and extending the shelf-life of food stored in the respective cooling chamber.

It may be worth stating that alternatives, by way of, but not limited to; shape and/or size, relative positioning and substitution of the preserving/deodorizing media are possible without deviation from the spirit and scope the invention.

Scope of Application

6.1 This invention titled ‘Active Scrub with Baking Soda for Fridge Freshening and Food Preservation’ can be adopted in any existing refrigerator/freezer/cooling cabinet, as also in those to be manufactured in future.

What is claimed is:

1. 7.1. A process, titled ‘Active Scrub’; which exploits the flow/momentum of the air emerging from the air vent of air circulation system of respective refrigerating appliance; to scrub such air with baking soda. Since environmental air of the cooling cabinet is a part the air contained in the air circulation system (FIG. 1.0), such scrubbing improves the efficacy of baking soda in deodorizing the environmental air and preservation of food stored in the cooling cabinet.
7.1.1 In the process ‘Active scrub’ claimed in 7.1, air emerging from the air vent of air circulation system of respective refrigerating appliance has momentum and thus impinges upon the baking soda kept/held across the direction of air flow (FIG. 4.1).
7.1.2 The process in claim 7.1 finds application in refrigerating appliances like refrigerators, freezers and cooling cabinets etc. having captive air circulation system and can be adopted in existing as well as yet to be manufactured units.
7.2 An attachment is envisaged to accomplish the process claimed in 7.1.
7.2.1 Attachment, as claimed in 7.2 consists of a perforated cartridge and complementary frame and fixture (FIGS. 3.1 and 3.2).
7.2.2 The attachment in claims 7.2 and 7.2.1 is positioned/fitted/fastened close to the air vent and oriented in such a way that cartridge lies across the direction of air flowing out of the air vent of air circulation system of the respective equipment (FIG. 4.1).
7.2.3 Baking Soda for scrubbing claimed in 7.1 is filled in the cartridge (FIG. 3.1) and fitted in the attachment (FIG. 3.3) claimed in 7.2 and 7.2.1.
7.2.3 Baking soda, referred to in claim 7.1 is in coarse form, like; but not limited to, crystals, granules, chunks, nuggets, tablets, pellets etc.
